(19) United States
(12) Patent Application Publication
(10) Pub. No.: US 2008/0033207 A1
(43) Pub. Date: Feb. 7, 2008

(54) Preparation of R-5- (2-(2-Ethoxyphenoxyetylamino) -2-Methoxybenzen-Sulphonamide Hydrochloride of High Chemical

(76) Inventors: Borut Furlan, Ljubljana (SI); Anton Copar, Smartno pri Litjii (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/584,651

(22) PCT Filed: Dec. 27, 2004
(86) PCT No.: PCT/SI04/00047

§ 371 Date: Apr. 17, 2007

(30) Foreign Application Priority Data
Dec. 29, 2003 (SI) P-200300319

(51) Int. Cl. C07C 311/16; C07C 303/40; C07C 303/44

(52) U.S. Cl. 564/86

(57) ABSTRACT

In the present invention, the process for the preparation of tamsulosin hydrochloride and subsequent purification with thermal crystallisation to provide essentially pure tamsulosin hydrochloride is disclosed.

### FIELD OF THE INVENTION

The present invention belongs to the field of chemical synthesis and relates to the synthesis of tamsulosin.

More particularly, this invention relates to processes for the preparation of tamsulosin and its purification to obtain pure tamsulosin hydrochloride.

### BACKGROUND OF THE INVENTION

Tamsulosin is a pharmaceutical active substance from the group of α₁-adrenergic receptor antagonists used in the treatment of functional disorders of the prostate. Chemically, tamsulosin belongs to benzenesulphonamides or sulphamoylphenetyl amine derivatives and is (R)-5-(2-(2-(2-ethoxyphenoxy)ethylamino)-1-propyl)-2-methoxybenzenesulphonamide (formula 1).

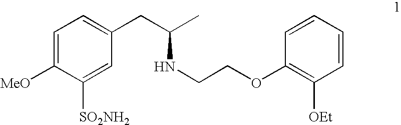

The preparation of 5-(2-(2-(2-ethoxyphenoxy)ethylamino)-1-propyl)-2-methoxy benzenesulphonamide, tamsulosin, as a racemic mixture of (R) and (S) enantiomers is described in EP34432.

Tamsulosin is commercially marketed in a form of the hydrochloride of pure (R)-enantiomer (1a) and is used for the treatment of benign prostatic hyperplasia.

In a process for the preparation of tamsulosin, it is desirable to arrive at an optical purity of more than 99% enantiomeric excess (also referred to as e.e.) on a final product or intermediate in as early stage of the synthesis as possible to avoid lengthy and costly purifications in later stages of the synthesis.

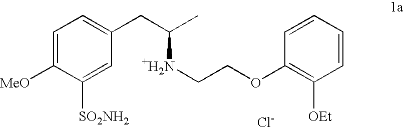

It is known to a skilled person that such high optical purity is hard to achieve and usually requires lengthy, laborious and complex enantiomer separation processes, resulting in low yields of the desired optically active product. Accordingly, commercially available intermediate compounds having a chiral centre, such as (R)-5-(2-amino-1-propyl)-2-methoxybenzenesulphonamide, having a high level of optical purity, e.g. greater than 90%, are generally more expensive than non optically pure analogs.

Preparation of the optically active compound (R)-5-(2-(2-(2-ethoxyphenoxy) ethylamino)-1-propyl)-2-methoxybenzenesulphonamide hydrochloride is disclosed in EP 380,144. Therein tamsulosin is prepared by a reaction of the optically active amine, (R)-5-(2-amino-1-propyl)-2-methoxybenzenesulphonamide (2), with the brominated ether, 1-(2-bromoethoxy)-2-ethoxybenzene (3). However the process of EP 380,144 requires the use of a molar excess of the optically active (R)-5-(2-amino-1-propyl)-2-methoxybenzenesulphonamide intermediate compound, which is also used as a base. Additionally the reaction process disclosed in EP 380,144 results inevitably in the formation of by-products and impurities, such that it is necessary to purify the crude product by column chromatography.

In CA 1,282,077 the reaction of the compounds (2) and (3) is disclosed whereby it is reported to be preferred to use equimolar amounts of the reagents if an external or additional base exists. The possibility of an excess amount of either compound has not been developed.

WO 03/35608 discloses a process wherein tamsulosin is produced by the reaction of the optically active amine of formula (2) with the brominated ether of formula (3) in the presence of an external base. According to WO 03/35608, the excess of the optically active reagent (2) required is reduced to a ratio of the reagents (2) and (3) of between 1:1 and 1:1.1. However, in the process of WO 03/35608 more expensive and ecologically less friendly solvents are used, such as dialkylamides, dialkylsulphoxides, N-methylpyrrolidone and sulpholane.

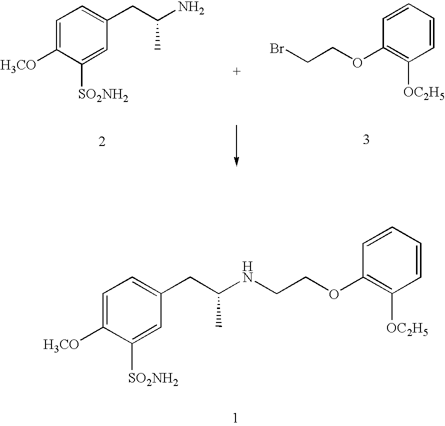

### SUMMARY OF THE INVENTION

In the first embodiment, the invention concerns tamsulosin hydrochloride comprising less than 0.1% of overalkylated products being bis-(2-(2-ethoxyphenoxy)ethyl substituted derivatives of 4-methoxy-3-sulphonamido benzenepropane-2-amine, wherein additional 2-(2-ethoxyphenoxy)ethyl substituents are bound to the sulphonamide nitrogen atom or propanamine nitrogen atom.

In another embodiment, the invention concerns a process for the preparation of tamsulosin hydrochloride comprising the reaction of R-5-(2-aminopropyl)-2-methoxybenzenesulphonamide with an excess of 1-(2-bromoethoxy)-2-ethoxybenzene in an organic solvent.

In another embodiment, the invention concerns a pharmaceutical formulation comprising such purified tamsulosin hydrochloride and other pharmaceutically acceptable excipients.

In another embodiment, the invention concerns the use of such purified tamsulosin hydrochloride for the preparation of a medicament for the treatment of benign prostatic hyperplasia.

### DETAILED DESCRIPTION OF THE INVENTION

It has been surprisingly found that the preparation of (R)-5-(2-(2-(2-ethoxy phenoxy)ethylamino)-1-propyl)-2-methoxybenzenesulphonamide from (R)-5-(2-amino-1-propyl)-2-methoxybenzenesulphonamide (2) and 1-(2-bromoethoxy)-2-ethoxybenzene (3) may be successfully accomplished without the need for the addition of any base by carrying out the reaction in the presence of a molar excess of the non-chiral reagent, 1-(2-bromoethoxy)-2-ethoxybenzene (3).

It has been found that in the presence of a molar excess of the non-chiral intermediate compound, 1-(2-bromoethoxy)-2-ethoxybenzene (3), the reaction equilibrium is moved towards the formation of tamsulosin even without the presence of an additional base.

Preferred solvents are lower alkyl alcohols, more preferred is methanol.

The excess of the reagent (3) over the reagent (2) is effective above the ratios of about 1.2:1 and may be increased to about 5:1, preferably to about 3:1. More preferred ratio is from about 1.5:1 to about 2:1, most preferred from about 1.7:1 to about 1.9:1.

The process for the production of tamsulosin according to the present invention allows the provision of a good yield of the crude product at a good level of purity. The product isolated directly from the reaction conversion may comprise about 75% to about 90% of tamsulosin hydrochloride. It has been surprisingly found that the expected overalkylation occurs only to a limited extent, such that the production process of the present invention provides a crude product of tamsulosin hydrochloride in which the contents of any one of the overalkylated products, e.g. N-(2-(2-ethoxyphenoxy)ethyl)-5-(2-(2-(2-ethoxyphenoxy)ethylamino)-1 -propyl)-2-methoxybenzenesulphonamide (5) or 5-(2-(bis-(2-(2-ethoxyphenoxy)ethyl)amino)-1-propyl)-2-methoxybenzenesulphonamide (4) or the excessed 1-(2-bromoethoxy)-2-ethoxybenzene (3) does not exceed 6%.

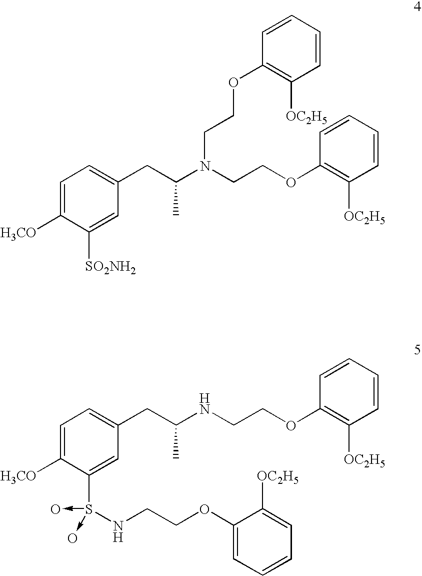

Tamsulosin hydrochloride can be obtained by treating tamsulosin base with ethanolic HCl.

The crude tamsulosin hydrochloride according to the present invention may comprise no more than 5% w/w, preferably no more than 3% w/w, of N-(2-(2-ethoxyphenoxy)ethyl)-5-(2-(2-(2-ethoxyphenoxy)ethylamino)-1-propyl)-2-methoxy benzenesulphonamide (5), no more than 6% w/w, preferably no more than 5% w/w, of 5-(2-(bis-(2-(2-ethoxyphenoxy)ethyl)amino)-1-propyl)-2-methoxybenzene sulphonamide (4), no more than 2% w/w, preferably no more than 1% w/w, of (R)-5-(2-amino-1-propyl)-2-methoxybenzenesulphonamide (2) and no more than 2% w/w, preferably no more than 1% w/w, of 1,2-bis(2-ethoxyphenoxy)ethane (6).

The contents of the overalkylated products in the crude product may be minimised whilst at the same time maintaining a high yield for the desired tamsulosin hydrochloride by adjusting the extent of the excess of the reagent (3). Preferably a ratio of reagents (2) to (3) of between about 1:1.5 to about 1:2, more preferably about 1:1.75 can be used. At this ratio, the yield of tamsulosin is still not essentially decreased but the contents of overalkylated products (4) and (5) may be reduced below 2%.

The crude product, obtained directly from the reaction process, may be additionally purified to yield tamsulosin having a pharmaceutically acceptable purity by using conventional purification methods, such as thermal recrystallisation whereby a solution of the product is heated to a higher temperature and then the mixture is cooled in order to recrystallise the product. Tamsulosin hydrochloride can be recrystallised by thermal recrystallisation from alcohols whereby a part of impurities is eliminated from the product.

Surprisingly, it has been found that for the purification of crude tamsulosin hydrochloride a high level of elimination of impurities is achieved where a mixture of ethanol and methanol is used as the recrystallisation solvent.

It has been found that a mixture with a higher proportion of ethanol removes better non-polar impurities whereas mixtures with a lower proportion of ethanol are more effective for removal of less polar overalkylated products. Ratios of methanol to ethanol of around 1:1 are preferred for the recrystallisation of tamsulosin hydrochloride. Ratios of about 1:1 have been shown to approximately evenly remove all impurities to a sufficiently low level and therefore has been identified as preferable taking into consideration also a better yield because the recovery of the product is somewhat greater with mixtures richer in ethanol.

According to another embodiment of the present invention, there is provided a process for the purification of tamsulosin hydrochloride comprising recrystallising tamsulosin from a solution in methanol or ethanol or a mixture of ethanol and methanol, by thermal recrystallisation. Preferably a mixture of methanol and ethanol is used in a ratio of methanol to ethanol of from about 7:3 to about 3:7, more preferably about 1:1 is used.

The process of the present invention allows for the production of tamsulosin hydrochloride of a high purity and at a good yield, even from starting materials which are not purified to a low content of impurities. For instance it has been found that although the starting compound, 1-(2-bromoethoxy)-2-ethoxybenzene (3), can contain up to about 8% of 1,2-bis(2-ethoxyphenoxy)ethane (6), according to the method of this invention, there is not more than 0.2% of said impurity in the final product.

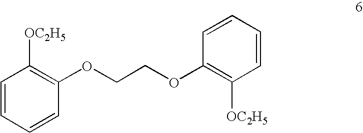

After purification of tamsulosin hydrochloride by recrystallisation from an ethanol/methanol mixture, tamsulosin hydrochloride having higher than 99.5% purity, even higher than 99.8% purity, may be obtained from, for example, tamsulosin hydrochloride having a purity of as low as 90%, even as low as 86%, after only two crystallisations.

Purification of tamsulosin hydrochloride by thermal recrystallisation according to the present invention allows the production of a purified product comprising as low as 0.08% w/w, even 0.06 % w/w of N,SO₂N-dialkylated products, i.e. N-(2-(2-ethoxyphenoxy)ethyl)-5-(2-(2-(2-ethoxyphenoxy)ethylamino)-1-propyl)-2-methoxy benzenesulphonamide (4) and less than 0.1% w/w of all overalkylated products.

The efficacy of purification in view of the invention enables that the process with an excess of the less expensive reagent (3) becomes an economical procedure for industrial production because in only two steps, a high quality pharmaceutical active substance can be obtained.

For the purification of crude tamsulosin hydrochloride to a high quality, it is preferable to carry out more than one, preferably two crystallisations. Where more than one crystallisation is effected, each crystallisation can be carried out in a different medium. Thus, for example, for somewhat poorer quality crude samples, it may be particularly effective to carry out the first crystallisation in the mixture of methanol to ethanol at a ratio of about 1:1 whereas the second one in a mixture with a higher proportion of methanol or in methanol alone.

The presence and contents of impurities have been proven with the standards of these compounds whereas by-products of the reactions, which are not simply available, are isolated from mother liquors by using preparative chromatography.

Tamsulosin hydrochloride obtained by the process according to the present invention is suitable for a pharmaceutical use in any pharmaceutical formulation whereby the crystals may be additionally milled to obtain particles of the size d(0.9) below 120 μm and d(0.5) below 50 μm.

Tamsulosin hydrochloride of the present invention in any pharmaceutical formulation can be then used for the treatment of benign prostatic hyperplasia.

### EXAMPLES

The present invention is illustrated but in no way limited by the following examples:

### Example 1

10 g (41 mmol) of 5-((R)-2-amino-1-propyl)-2-methoxybenzenesulphonamide, 19 g (77 mmol) of 2-(2-ethoxyphenoxy)ethylbromide and 170 ml of methanol are heated under reflux for 43 hours. Methanol is evaporated under vacuum on a rotavapor at 60° C. To the residue, 170 ml of water and 130 ml of ethyl acetate are added and while cooling and stirring also 16 g of 50% aqueous sodium hydroxide. After separation of both phases, the water phase is extracted twice with 100 ml of ethyl acetate. Combined extracts are washed twice with 130 ml of water and evaporated in vacuo on a rotavapor at 60° C. The residue is dissolved in 100 ml of ethanol and while cooling and stirring, 7 ml of ethanolic hydrogene chloride solution (300 mg HCl/ml) is added. While cooling (0° C.), the mixture is stirred for 4 hours and the formed crude (−)-(R)-5-(2-(2-(2-ethoxyphenoxy)ethylamino)-1-propyl)-2-methoxy benzenesulphonamide hydrochloride (TH) is filtered, washed with 20 ml of cooled ethanol (of about 0° C.) and dried under vacuum at 40° C. 7.0 g of a crude product is obtained.

HPLC analysis:

| | |
|---|---|
| (−)-(R)-5-(2-(2-(2-ethoxyphenoxy)ethylamino)-1-propyl)-2- | 78.0% |
| methoxybenzenesulphonamide hydrochloride | |
| 5-((R)-2-amino-1-propyl)-2-methoxybenzenesulphonamide (2) | 0.8% |
| 2-(2-ethoxyphenoxy)ethylbromide (3) | 0.8% |
| N-(2-(2-ethoxyphenoxy)ethyl)-5-(2-(2-(2- | 4.2% |
| ethoxyphenoxy)ethylamino)-1-propyl)-2- | |
| methoxybenzenesulphonamide | |
| 5-(2-(bis-(2-(2-ethoxyphenoxy)ethyl)ethyl)amino)-1-propyl)-2- | 5.9% |
| methoxybenzenesulphonamide | |
| 1,2-bis(2-ethoxyphenoxy)ethane (6) | 7.9% |

### Example 2

200 g (0.82 mol) of 5-((R)-2-amino-1-propyl)-2-methoxybenzenesulphonamide, 350 g (1.43 mol) of 2-(2-ethoxyphenoxy)ethylbromide and 3.4 l of methanol are heated under reflux for 45 hours. Methanol is evaporated in vacuo on a rotavapor at 60° C. To the residue 3.4 l of water and 2.6 l of ethyl acetate are added and while cooling and stirring also 650 g of 50% aqueous sodium hydroxide. After separation of both phases, the water phase is extracted twice with 2 l of ethyl acetate. The combined extracts are washed twice with 2.6 l of water and evaporated in vacuo on a rotavapor at 60° C. The residue is dissolved in 2 l of ethanol and 140 ml of ethanolic hydrogen chloride solution (300 mg HCl/ml) is added while cooling and stirring. While cooling (0° C.) the mixture is stirred for 4 hours and the formed crude (−)-(R)-5-(2-(2-(2-ethoxyphenoxy)ethylamino)-1-propyl)-2-methoxybenzenesulphonamide hydrochloride (TH) is filtered, washed with 400 ml of cool ethanol and dried in vacuo at 40° C. 158.0 g of a crude product is obtained.

HPLC analysis:

| | |
|---|---|
| (−)-(R)-5-(2-(2-(2-ethoxyphenoxy)ethylamino)-1-propyl)-2- | 86.1% |
| methoxybenzenesulphonamide hydrochloride | |
| 5-((R)-2-amino-1-propyl)-2-methoxybenzenesulphonamide | 1.53% |
| 2-(o-ethoxyphenoxy)ethylbromide | 2.84% |
| N-(2-(2-ethoxyphenoxy)ethyl)-5-(2-(2-(2- | 1.79% |
| ethoxyphenoxy)ethylamino)-1-propyl)-2- | |
| methoxybenzenesulphonamide | |
| 5-(2-(bis-(2-(2-ethoxyphenoxy)ethyl)amino)-1-propyl)-2- | 0.98% |
| methoxybenzenesulphonamide | |
| 1,2-bis(2-ethoxyphenoxy)ethane (6) | 6.17% |

### Example 3

10 g of (−)-(R)-5-(2-(2-(2-ethoxyphenoxy)ethylamino)-1-propyl)-2-methoxybenzene sulphonamide hydrochloride (TH) from Example 2 is recrystallised from mixtures of methanol and ethanol.

Analysis:

| Meth- | | | | |
|---|---|---|---|---|
| anol | | Quantity | | |
| to | HPLC-composition | of | | |
| ethanol | of the starting raw | solvent | | HPLC-composition |
| ratio | material* | used | Yield | of the product* |
| 100:0 | TH 86.1% | 120 ml | 7.77 g | TH 95.84% |
| | impurity (2) 1.53% | | (77.7%) | impurity (2) 0.09% |
| | impurity (3) 2.84% | | | impurity (3) 0.0% |
| | impurity (4) 1.79% | | | impurity (4) 0.24% |
| | impurity (5) 0.98% | | | impurity (5) 0.05% |
| | impurity (6) 6.17% | | | impurity (6) 3.73% |
| 90:10 | TH 86.1% | 140 ml | 7.75 g | TH 95.5% |
| | impurity (2) 1.53% | | (77.5%) | impurity (2) 0.12% |
| | impurity (3) 2.84% | | | impurity (3) 0.0% |
| | impurity (4) 1.79% | | | impurity (4) 0.31% |
| | impurity (5) 0.98% | | | impurity (5) 0.08% |
| | impurity (6) 6.17% | | | impurity (6) 3.94% |
| 70:30 | TH 86.1% | 210 ml | 7.78 g | TH 95.9% |
| | impurity (2) 1.53% | | (77.8%) | impurity (2) 0.12% |
| | impurity (3) 2.84% | | | impurity (3) 0.0% |
| | impurity (4) 0.98% | | | impurity (4) 0.31% |
| | impurity (5) 1.79% | | | impurity (5) 0.08% |
| | impurity (6) 6.17% | | | impurity (6) 3.49% |
| 50:50 | TH 86.1% | 340 ml | 7.41 g | TH 99.27% |
| | impurity (2) 1.53% | | (74.1%) | impurity (2) 0.15% |
| | impurity (3) 2.84% | | | impurity (3) 0.0% |
| | impurity (4) 0.98% | | | impurity (4) 0.32% |
| | impurity (5) 1.79% | | | impurity (5) 0.08% |
| | impurity (6) 6.17% | | | impurity (6) 0.0% |
| 30:70 | TH 86.1% | 500 ml | 7.55 g | TH 99.28% |
| | impurity (2) 1.53% | | (75.5%) | impurity (2) 0.17% |
| | impurity (3) 1.79% | | | impurity (3) 0.0% |
| | impurity (4) 0.98% | | | impurity (4) 0.32% |
| | impurity (5) 1.79% | | | impurity (5) 0.10% |
| | impurity (6) 6.17% | | | impurity (6) 0.0% |

*impurity (2) = 5-((R)-2-amino-1-propyl)-2-methoxybenzenesulphonamide impurity (3) = 2-(2-ethoxyphenoxy)ethylbromide impurity (4) = N-(2-(2-ethoxyphenoxy)ethyl)-5-(2-(2-(2-ethoxyphenoxy) ethyl)amino)-1-propyl)-2-methoxybenzenesulphonamide impurity (5) = 5-(2-(bis-(2-(2-ethoxyphenoxy)ethyl) amino)-1-propyl)-2-methoxybenzenesulphonamide impurity (6) = 1,2-bis(2-ethoxyphenoxy)ethane (6)

### Example 4

7.0 g of recrystallised (−)-(R)-5-(2-(2-(2-ethoxyphenoxy)ethylamino)-1-propyl)-2-methoxybenzenesulphonamide hydrochloride (TH) from Example 3 is recrystallised from mixtures of methanol and ethanol.

Analysis:

| Meth- | | | | |
|---|---|---|---|---|
| anol | HPLC- | | | |
| to | composition of | Quantity | | HPLC- |
| ethanol | the starting raw | of solvent | | composition of |
| ratio | material* | used | Yield | the product* |
| 100:0 | TH 95.84% | 90 ml | 5.44 g | TH 97.77% |
| | impurity (2) 0.09% | | (77.7%) | impurity (2) 0.0% |
| | impurity (3) 0.0% | | | impurity (3) 0.0% |
| | impurity (4) 0.24% | | | impurity (4) 0.04% |
| | impurity (5) 0.05% | | | impurity (5) 0.0% |
| | impurity (6) 3.73% | | | impurity (6) 2.19% |
| 90:10 | TH 95.5% | 110 ml | 5.64 g | TH 97.53% |
| | impurity (2) 0.12% | | (80.6%) | impurity (2) 0.12% |
| | impurity (3) 0.0% | | | impurity (3) 0.0% |
| | impurity (4) 0.31% | | | impurity (4) 0.06% |
| | impurity (5) 0.08% | | | impurity (5) 0.00% |
| | impurity (6) 3.94% | | | impurity (6) 2.41% |
| 70:30 | TH 95.9% | 160 ml | 5.70 g | TH 99.89% |
| | impurity (2) 0.12% | | (81.4%) | impurity (2) 0.0% |
| | impurity (3) 0.0% | | | impurity (3) 0.0% |
| | impurity (4) 0.31% | | | impurity (4) 0.05% |
| | impurity (5) 0.08% | | | impurity (5) 0.0% |
| | impurity (6) 3.49% | | | impurity (6) 0.0% |
| 50:50 | TH 99.27% | 230 ml | 5.95 g | TH 99.85% |
| | impurity (2) 0.15% | | (85.0%) | impurity (2) 0.0% |
| | impurity (3) 0.0% | | | impurity (3) 0.0% |
| | impurity (4) 0.32% | | | impurity (4) 0.06% |
| | impurity (5) 0.08% | | | impurity (5) 0.0% |
| | impurity (6) 0.0% | | | impurity (6) 0.0% |
| 30:70 | TH 99.28% | 340 ml | 5.98 g | TH 99.81% |
| | impurity (2) 0.17% | | (85.4%) | impurity (2) 0.02% |
| | impurity (3) 0.0% | | | impurity (3) 0.0% |
| | impurity (4) 0.32% | | | impurity (4) 0.08% |
| | impurity (5) 0.10% | | | impurity (5) 0.0% |
| | impurity (6) 0.0% | | | impurity (6) 0.0% |

*impurity (2) = 5-((R)-2-amino-1-propyl)-2-methoxybenzenesulphonamide impurity (3) = 2-(2-ethoxyphenoxy)ethylbromide impurity (4) = N-(2-(2-ethoxyphenoxy)ethyl)-5-(2-(2-(2-ethoxyphenoxy) ethyl)amino)-1-propyl)-2-methoxybenzenesulphonamide impurity (5) = 5-(2-(bis-(2-(2-ethoxyphenoxy)ethyl) amino)-1-propyl)-2-methoxybenzenesulphonamide impurity (6) = 1,2-bis(2-ethoxyphenoxy)ethane (6)

### Example 5

7.0 g of (−)-(R)-5-(2-(2-(2-ethoxyphenoxy)ethylamino)-1-propyl)-2-methoxybenzene sulphonamide hydrochloride (TH) from Example 1 is recrystallised from the 1:1 methanol/ethanol mixture, the product is dried at 40° C. in vacuo and recrystallised again from methanol. 4.61 g of the product is obtained. The product is milled using a hammer-type mill at 4800 rpm.

Analysis:

| HPLC-composition | HPLC-composition | |
|---|---|---|
| of the | of the once | HPLC-composition of the |
| starting raw material* | crystallised product* | twice crystallised product* |
| TH 78.0% | TH 95.84% | TH 97.77% |
| impurity (2) 0.8% | impurity (2) 0.09% | impurity (2) 0.0% |
| impurity (3) 0.8% | impurity (3) 0.0% | impurity (3) 0.0% |
| impurity (4) 4.2% | impurity (4) 0.24% | impurity (4) 0.04% |
| impurity (5) 5.9% | impurity (5) 0.05% | impurity (5) 0.0% |
| impurity (6) 7.9% | impurity (6) 3.73% | impurity (6) 2.19% |

*impurity (2) = 5-((R)-2-amino-1-propyl)-2-methoxybenzenesulphonamide impurity (3) = 2-(2-ethoxyphenoxy)ethylbromide impurity (4) = N-(2-(2-ethoxyphenoxy)ethyl)-5-(2-(2-(2-ethoxyphenoxy) ethyl)amino)-1-propyl)-2-methoxybenzenesulphonamide impurity (5) = 5-(2-(bis-(2-(2-ethoxyphenoxy)ethyl) amino)-1-propyl)-2-methoxybenzenesulphonamide impurity (6) = 1,2-bis(2-ethoxyphenoxy)ethane (6)

### Particle size analysis (Malvern): d (90)=113.7 μm; d (50)=31.3 μm.

### Example 6

The filtrate obtained after filtration of the product from Example 2 from the methanol to ethanol ratio 50:50 is evaporated and the residue in 2-g-aliquots is applied onto the column 200×50 mm with the stationary phase Luna 1 μM, prep C18(2), and eluted with the mobile phase (5 ml/l triethylamine, pH up to 2.8 with orthophosphoric acid, 20% methanol) at a flow rate 150 ml/min. Two fractions of each batch are collected, the corresponding fractions from different batches are combined, methanol evaporated, desalted, concentrated and lyophilized. The solid fractions A and B in the quantitative ratio 1:1.5 are obtained.

Fraction A:

5-(2-(bis-(2-(2-ethoxyphenoxy)ethyl)amino)-1-propyl)-2-methoxybenzenesulphonamide (5)

Appearance: Hygroscopic white crystals.

MS: 573 (M+H)⁺

NMR (300 MHz, TMS, CD₃OD); δ (ppm): 6.8-7.8 (11H, m, aromatic protons); 3.80-4.10 (8H, m, OCH₂); 3.87 (8H, S, OCH₃); 2.40-3.20 (7H, m, CH₂N, CH₂CHN); 1.30 (6H, t, OCH₂CH₃); 1.05 (3H, d, CHCH₃).

Fraction B:

N-(2-(2-ethoxyphenoxy)ethyl)-5-(2-(2-(2-ethoxyphenoxy)ethylamino)-1-propyl)-2-methoxybenzenesulphonamide (6)

Appearance: White crystals.

MS: 573 (M+H)⁺

NMR (300 MHz, TMS, CD₃OD); δ (ppm): 6.7-7.8 (11H, m, aromatic protons); 3.80-4.30 (8H, m, OCH₂); 3.86 (8H, S, OCH₃); 2.60-3.30 (7H, m, CH₂N, CH₂CHN); 1.35 and 1.38 (6H, t, t, OCH₂CH₃); 1.15 (3H, d, CHCH₃).

### Example 7

Bis-(2-ethoxyphenoxy)ethane is isolated from a commercial raw material, 2-(2-ethoxyphenoxy)ethylbromide, by column chromatography on silica gel (ether: petroleum ether=1:2 v/v).

1. Tamsulosin hydrochloride characterised in that it comprises less than 0.1% of overalkylated products.

2. Tamsulosin hydrochloride according to claim 1 wherein said overalkylated products are bis-(2-(2-ethoxyphenoxy)ethyl substituted derivatives of 4-methoxy-3-sulphonamidobenzenepropane-2-amine wherein additional (2-(2-ethoxyphenoxy) ethyl substituents are bound to the sulphonamide nitrogen atom or propanamine nitrogen atom.

3. Tamsulosin hydrochloride according to claim 1 characterised in that it comprises less than 0.02% of 5-(2-(bis-(2-(2-ethoxyphenoxy)ethyl)amino)propyl)-2-methoxybenzenesulphonamide.

4. Tamsulosin hydrochloride according to claim 1 characterised in that it comprises less than 0.06% of N-(2-(2-ethoxyphenoxy)ethyl)-5-(2-(2-(2-ethoxyphenoxy)ethylamino)propyl)-2-methoxybenzenesulphonamide.

5. A process for the preparation of tamsulosin hydrochloride characterised in that it comprises the reaction of R-5-(2-aminopropyl)-2-methoxybenzenesulphonamide with an excess of 1-(2-bromoethoxy)-2-ethoxybenzene in an organic solvent.

6. The process for the preparation of tamsulosin hydrochloride according to claim 5 wherein the excess of 1-(2-bromoethoxy)-2-ethoxybenzene is from about 1.2 to about 3.

7. The process for the preparation of tamsulosin hydrochloride according to claim 5 wherein said organic solvent is methanol.

8. A process for the purification of tamsulosin hydrochloride comprising recrystallising tamsulosin hydrochloride from a solution in methanol or ethanol or a mixture of ethanol and methanol by thermal recrystallisation.

9. A process for the purification of tamsulosin hydrochloride with less than about 90% of the active substance wherein the contents of the active substance above 99.8% is achieved with at most two thermal crystallisations from a mixture of methanol and ethanol.

10. The process for the purification of tamsulosin hydrochloride according to claim 8 wherein recrystallisation is carried out from a mixture of methanol and ethanol in a ratio of between about 3:7 and about 7:3.

11. (canceled)

12. (canceled)

13. (canceled)

14. (canceled)
